(19) United States
(12) Patent Application Publication
(10) Pub. No.: US 2007/0056859 A1
(43) Pub. Date: Mar. 15, 2007

(54) Efficacious scalp health predictor

(76) Inventors: Faiz Feisal Sherman, West Chester, OH (US); Vladimir Gartstein, Cincinnati, OH (US); David Burton Moore, Hamilton, OH (US); Carl Hinz Margraf III, Frankfurt am Main (DE); Brian Keith Fisher, Cincinnati, OH (US)

Correspondence Address: THE PROCTER & GAMBLE COMPANY;INTELLECTUAL PROPERTY DIVISION, WINTON HILL BUSINESS CENTER - BOX 161, 6110 CENTER HILL AVENUE, CINCINNATI, OH, 45224, US

(21) Appl. No.: 11/515,142

(22) Filed: Sep. 1, 2006

Related U.S. Application Data
(60) Provisional application No. 60/713,864, filed on Sep. 2, 2005.

(51) Int. Cl. C12Q 1/00

(52) U.S. Cl. 205/777.5

(57) ABSTRACT

A device is provided for measuring the moisture content of skin, such as the scalp. The device comprises (a) a body portion containing a sensor circuit therein, said sensor circuit operable to generate a signal correlated to the moisture content of said scalp, said body portion having a first color; (b) a removable cover attached to said body portion, said cover having a second color; and (c) said first color and said second color match a color scheme of a personal care product thereby signaling at least one performance characteristic of said device.

### CROSS REFERENCE TO RELATED APPLICATIONS

This application claims the benefit of U.S. Provisional application Ser. No. 60/713,864, filed on Sep. 2, 2005.

### FIELD OF THE INVENTION

The present invention relates to a device for measuring the moisture content of the scalp. Efficacy and credibility of the device is communicated by means of using the existing color scheme and labeling associated with a brand of hair care products.

### BACKGROUND OF THE INVENTION

In the hair care field it is often beneficial to assess the condition of a consumer's scalp. Devices for measuring the moisture content have been developed in the past to determine the moisture level of skin or hair. Proper selection of a device requires explicit labeling and/or instructions in order to convey a message regarding proper selection and usage. This is accomplished via printed text, pictures, diagrams or labels.

However, none of these devices combine the color schemes and labeling associated with a brand of hair care products in order to communicate that the device carries the same efficacy and credibility that the brand of hair care product has established.

### SUMMARY OF THE INVENTION

In one embodiment, the present invention is directed to a device for measuring the moisture content of the scalp. The device comprises: (a) a body portion containing a sensor circuit therein, the sensor circuit operable to generate a signal correlated to the moisture content of the scalp, the body portion having a first color; (b) a removable cover attached to the body portion, the cover having a second color; and (c) the first color and the second color match a color scheme of a personal care product thereby signaling at least one performance characteristic of the device.

In another embodiment, the present invention is directed to a device for measuring the moisture content of the scalp comprising: (a) a body portion containing a sensor circuit therein, the sensor circuit operable to generate a signal correlated to the moisture content of the scalp; (b) at least one label attached to the body portion; and (c) indicia disposed on the at least one label, the indicia relates to at least one performance characteristic of said device.

In yet another embodiment, the present invention is directed to a device for measuring the moisture content of the skin comprising: (a) a body portion containing a sensor circuit therein, the sensor circuit operable to generate a signal correlated to the moisture content of the skin; (b) a removable cover attached to the body portion; and (c) the body portion and the cover are the same color, the color matching a color scheme of a personal care product thereby signaling at least one performance characteristic of the device.

These and other features, aspects and advantages of the present invention will become evident to those skilled in the art from a reading of the present disclosure.

### BRIEF DESCRIPTION OF THE DRAWINGS

While the specification concludes with claims which particularly point out and distinctly claim the invention, it is believed the present invention will be better understood from the following description taken in conjunction with the accompanying drawings.

FIG. 1 is the color line for white and blue; and

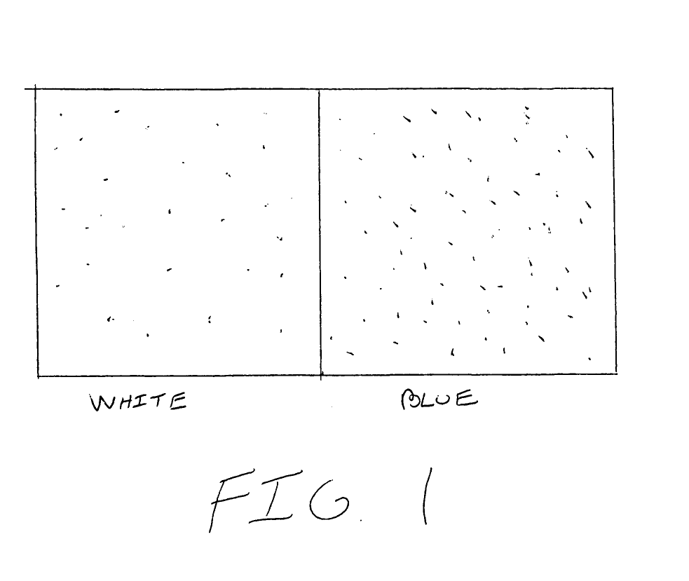

FIG. 2 is a view of a device in accordance with one embodiment of the present invention.

### DETAILED DESCRIPTION OF THE INVENTION

According to the present invention, devices are described for measuring the moisture content of the scalp. The determination of the moisture content in skin, including scalp, is used to quantify various physical and cosmetic characteristics of the skin. For example, a scalp with a low moisture content is unhealthy and may exhibit signs of dandruff, including flakes, dryness, tightness, itchiness and/or redness/irritation. Skin hydration is a function of its normal biological activity that results in continuous moisture flux from within the body to the environment. The improvement of skin barrier function results in greater skin hydration and less moisture loss. As a result, the physical and cosmetic characteristics of skin may be improved, for example, with treatments that restore skin to normal conditions and improve its barrier function. Improvement of barrier function, in turn, also results in protecting the skin from environmental, physical, chemical, or biological insults and results in an overall improvement in skin health.

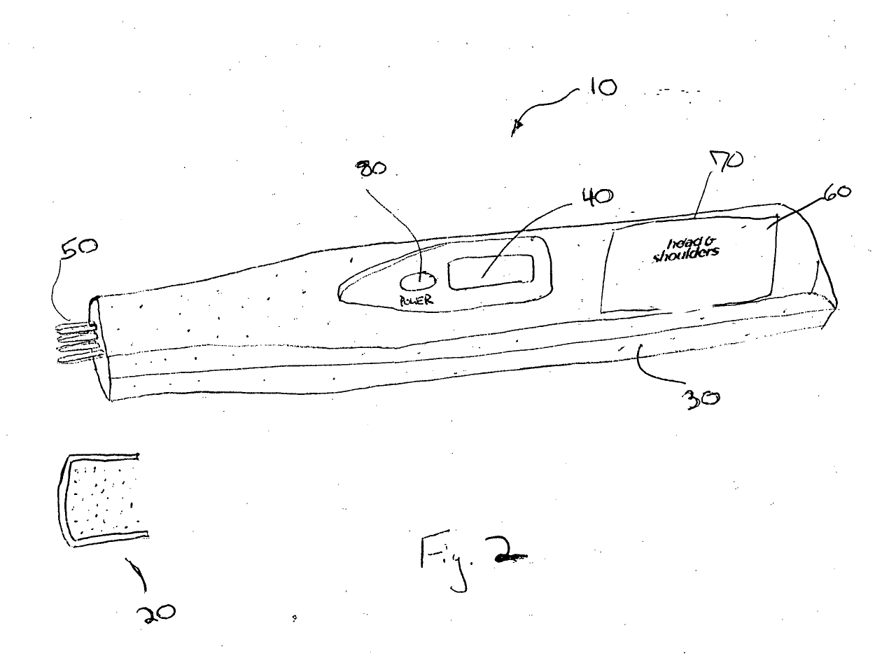

A device according to the present invention is exemplified in FIG. 2. Any suitable device for measuring moisture content may be used. For example, an electronic device comprising an impedance sensor, as described in U.S. Patent Application entitled “Method and Device for Indicating Moisture Content of Skin” filed on Sep. 2, 2005, Attorney Docket No. 10121P, can be used to measure the moisture content of the scalp. Suitable devices for measuring moisture content also include radio frequency, infrared, nuclear magnetic resonance, mechanical vibration, skin deformation, iontophoresis, topology, friction, trans epidermal water loss (TEWL), optical and heat dissipation.

In one embodiment, device 10 is shown as elongate in shape and is designed so that it is suitable for use in the hand. In one example, device 10 is provided in at least two separable parts, a removable cover 20 and a body portion/housing 30. Cover 20 and body portion 30 may be made from any suitable polymers.

The body portion 30 also contains a sensor circuit operable to generate a signal correlated to the moisture content of the scalp. In one embodiment, the sensor circuit of the device 10 operates in a frequency range between about 100 KHz and about 3.0 MHz, although other frequency ranges are possible as well. For example, in the measurement of the moisture content of the scalp, the device 10 of the present invention operates under the principle that as the moisture content of skin increases, its effective relative electrical impedance decreases that result in reduction sensor circuit frequency. The device 10 is designed to measure the skin electrical impedance, and from that measurement, the moisture content of the skin can be determined. The moisture content value may be presented on a visual display 40, for example, an LED display. One end of the body portion 30 has a plurality of pins 50 extending therefrom. In one embodiment, pins 50 are made of or coated with inert well conducting material, for example, gold. In one embodiment, the pins range in length from about 5 mm to about 30 mm. In another embodiment, the pins are from about 10 mm to about 20 mm in length. In yet another embodiment, the pins are from about 15 mm to about 20 mm in length. Power to the device 10 can be manually turned off using interface component 80, here, shown as an ON/OFF button. In some implementations, other component 80 types may be used such as a sliding switch or a toggle switch.

The present invention also relates to particular color schemes for use with the device 10. The term “color,” as used herein, relates to the phenomenon of visual perception that enables one to differentiate otherwise identical objects. In one embodiment, a color scheme is chosen for the device 10 that matches a color scheme of a personal care product. Exemplary personal care products, may include, without limitation, lotions, creams, gels, tonics, after shave, sticks, sprays, ointments, pastes, powders, mousse, shampoos, conditioners, oils, colorants, and biomedical and dermatological treatments. In one example, the cover 20 has a first color, for example, blue, and the body portion 30 has a second color, for example, white. As a result, the color scheme of the device 10 matches the color scheme of a hair care product, for example, HEAD & SHOULDERS® shampoo. In another example, cover 20 and body portion 30 are the same color, for example, blue. Moreover, the use of a color scheme that matches a color scheme of a personal care product is used to communicate or signal device performance characteristics.

A device's performance characteristic is the recognition of the execution of a particular characteristic of the device. As used herein, the term “characteristic” refers to any distinguishing trait, quality, or properties of the device. Nonlimiting examples of performance characteristics include, anti-dandruff efficacy, dry scalp efficacy, scalp care, dry skin benefits and skin cosmetic benefits. In another embodiment, the device 10 includes at least one label 60 attached to the body portion 30. The label 60 may include indicia 70 disposed thereon and relating to at least one performance characteristic of the device 10. The term “label,” as used herein, relates to the tangible medium on which indicia are expressed, including, for example, the placing of indicia directly on the device 10 (for example, printing or molding) or the printing of indicia on a substrate wherein the substrate is placed on the outside surface of the device 10. The term “indicia,” as used herein, relates to identifying markings, which may include, for example, words and/or graphics. The indicia element is chosen from a visual indicator or a narrative indicator. In one embodiment, the device of the present invention includes at least one indicia element and in another embodiment the device includes two or more indicia elements.

The term “visual indicator,” as used herein, includes any illustration, painting, photograph, drawing, picture, logo, hologram or scalp shot, that visually communicates or signals device performance characteristics. Nonlimiting examples of visual indicators include: orb, swirl, scribble, embossing, swoosh and arc.

The term “narrative indicator,” as used herein, includes letters, numbers or combination thereof that communicate or signal device performance characteristics. Nonlimiting examples of narrative indicators include: “head & shoulders” (text or stylized form) and “h & s” (text or stylized form).

In one embodiment, the color of the device may compliment the visual indicator and/or narrative indicator, thereby communicating or signaling device performance characteristics of the device.

### EXAMPLE

The following example is given solely for the purpose of illustration and is not to be construed as a limitation of the present invention, as many variations of the invention are possible without departing from the spirit and scope of the invention.

### Example 1

In order to determine a correlation between appearance and performance characteristics, a broad test was conducted wherein participants were shown a series of pictures and asked to fill out a questionnaire. Exemplary results as shown below in Table I.

TABLE I

| | Current Head & | Head & Shoulders ® | Head & Shoulders ® |
|---|---|---|---|
| | Shoulders ® bottle with | bottle with white body | bottle with white |
| | white body and blue | and blue cap having | bottle and blue cap |
| | cap having graphics | graphics containing | having graphics |
| | containing the H & S | modified H & S logo and | containing the H & S |
| | logo and Head & | Head & Shoulders ® | logo and no Head & |
| | Shoulders ® brand name | brand name | Shoulders ® brand name |
| Recognition of Head & | | | |
| Shoulders ® Brand (%) | | | |
| a) Strategic Target* | 94 | 79 | 64 |
| b) H&S Users** | 93 | 94 | 79 |
| b) Non H&S Users*** | 96 | 61 | 50 |
| Agree Product Will | | | |
| Treat Dandruff | | | |
| (Dandruff Efficacy) (%) | | | |
| a) Strategic Target | 86 | 79 | 84 |
| b) H&S Users | 85 | 86 | 93 |
| c) Non H&S Users | 87 | 71 | 75 |

*Consumers that indicated that they have a scalp condition, for example, dandruff
**Consumers from the Strategic Target that use Head & Shoulders ® brand shampoo
***Consumers from the Strategic Target that do not use Head & Shoulder ® brand shampoo

All documents cited in the Detailed Description of the Invention are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A device for measuring the moisture content of the scalp, said device comprising:
a) a body portion containing a sensor circuit therein, said sensor circuit operable to generate a signal correlated to the moisture content of said scalp, said body portion having a first color;
b) a removable cover attached to said body portion, said cover having a second color; and
c) said first color and said second color match a color scheme of a personal care product thereby signaling at least one performance characteristic of said device.

2. The device according to claim 1, wherein said sensor circuit operates in a frequency range between about 100 KHz and about 3.0 MHz.

3. The device according to claim 1, wherein said first color and second color are selected from the group consisting of white and blue.

4. The device according to claim 3, wherein said first color is white.

5. The device according to claim 3, wherein said second color is blue.

6. The device according to claim 1, wherein said personal care product is selected from the group consisting of lotions, creams, gels, tonics, after shave, sticks, sprays, ointments, pastes, powders, mousse, shampoos, conditioners, oils, colorants, and biomedical and dermatological treatments.

7. The device according to claim 1, wherein said at least one performance characteristic is anti-dandruff efficacy.

8. The device according to claim 1, wherein said at least one performance characteristic is dry scalp efficacy.

9. A device for measuring the moisture content of the scalp, said device comprising:
a) a body portion containing a sensor circuit therein, said sensor circuit operable to generate a signal correlated to the moisture content of said scalp;
b) at least one label attached to said body portion; and
c) indicia disposed on said at least one label, said indicia relates to at least one performance characteristic of said device.

10. The device according to claim 9, wherein said indicia is a visual indicator.

11. The device according to claim 9, wherein said indicia is a narrative indicator.

12. The device according to claim 9, wherein said at least one performance characteristic is anti-dandruff efficacy.

13. The device according to claim 9, wherein said at least one performance characteristic is dry scalp efficacy.

14. A device for measuring the moisture content of skin, said device comprising:
a) a body portion containing a sensor circuit therein, said sensor circuit operable to generate a signal correlated to the moisture content of said skin;
b) a removable cover attached to said body portion; and
c) said body portion and said cover are the same color, said color matching a color scheme of a personal care product thereby signaling at least one performance characteristic of said device.

15. The device according to claim 14, wherein said sensor circuit operates in a frequency range between about 100 KHz and about 3.0 MHz.

16. The device according to claim 14, wherein said color is blue.

17. The device according to claim 14, wherein said personal care product is selected from the group consisting of lotions, creams, gels, tonics, after shave, sticks, sprays, ointments, pastes, powders, mousse, shampoos, conditioners, oils, colorants, and biomedical and dermatological treatments.

18. The device according to claim 14, wherein said at least one performance characteristic is anti-dandruff efficacy.

19. The device according to claim 14, further comprising at least one label attached to said body portion, said label having indicia disposed thereon.
